(19) United States
(12) Patent Application Publication
(10) Pub. No.: US 2014/0084028 A1
(43) Pub. Date: Mar. 27, 2014

(54) WEARABLE SANITIZING AGENT DISPENSER

(71) Applicant: Christopher Gunn, Orlando, FL (US)

(72) Inventor: Christopher Gunn, Orlando, FL (US)

(21) Appl. No.: 13/625,457

(22) Filed: Sep. 24, 2012

(51) Int. Cl. B65D 83/00

(52) U.S. Cl. 222/175; 222/183

(57) ABSTRACT

A wearable sanitizing dispenser device includes a holster having a hingedly connected front half and rear half that define an interior space. A dispenser located within the interior space includes a canister connector, a dispensing valve, a control button and an output chamber. A wearable sanitizing dispenser device also includes a holster having a hingedly connected front half and rear half that define an interior space. A removable canister housing a sanitizing agent stored within the interior space, and a dispenser located within the interior space includes a canister connector, a dispensing valve, a control button and an output chamber.

### CROSS-REFERENCE TO RELATED APPLICATIONS

This application claims the benefit of Provisional U.S. Application Ser. No. 61/539,127 filed on Sep. 26, 2011, the contents of which are incorporated herein by reference.

### TECHNICAL FIELD

The present invention relates generally to sanitizer dispensing units, and more particularly to a device capable of dispensing a sanitizing agent while storing the same in a sturdy container that can be comfortably worn on an individual during normal activities.

### BACKGROUND

The statements in this section merely provide background information related to the present disclosure and may not constitute prior art.

The average person touches dirty items such as door handles, money and the like hundreds of times each day. However, it is well known that the most common means of transmitting contagions such as bacteria and diseases comes from direct physical contact between individuals. This is especially worrisome for public service members and first responders such as police officers, EMT's, and firemen, for example, for whom physical contact with members of the public is part of the job description.

Although, it is standard practice for first responders such as EMT's and firemen to wear gloves and other protective items before touching a member of the public, police officers are not always afforded such a luxury, as a casual encounter can turn into a violent struggle in an instant.

To this end, many first responders and public service individuals keep bottles of hand sanitizer within their vehicles in an attempt to stymie the progress and growth of bacteria on their hands after each contact with a member of the public. Unfortunately however, it is not uncommon for first responders to become separated from their vehicle for extended periods of time, thereby making the hand sanitizers unavailable when they are needed most.

Accordingly, there remains a need for a device capable of storing and dispensing sanitizing agents that can be worn on an individual taking part in the daily requirements of their job.

### SUMMARY OF THE INVENTION

The present invention is directed to a wearable sanitizing dispenser device. One embodiment of the present invention can include a holster having a hingedly connected front half and rear half that define an interior space for receiving a removable cartridge containing a sanitizing agent. A dispenser having a canister connector, a dispensing valve, a control button and an output chamber is positioned within the interior space to discharge the sanitizing agent to a device user.

Another embodiment of the present invention can include padding and thermal insulation for protecting an inserted cartridge from damage caused by physical impact and extreme temperature variations.

Yet another embodiment of the present invention can include a holster having a unitary construction and housing a permanent canister.

This summary is provided merely to introduce certain concepts and not to identify key or essential features of the claimed subject matter.

### BRIEF DESCRIPTION OF THE DRAWINGS

Presently preferred embodiments are shown in the drawings. It should be appreciated, however, that the invention is not limited to the precise arrangements and instrumentalities shown.

FIG. 1 is a perspective view of a wearable sanitizing dispenser device in a closed position, in accordance with one embodiment of the invention.

FIG. 2 is a perspective view of the wearable sanitizing dispenser device in an open position, in accordance with one embodiment of the invention.

FIG. 3 is a bottom view of the wearable sanitizing dispenser device, in accordance with one embodiment of the invention.

FIG. 4 is a perspective view of the wearable sanitizing dispenser device in operation, in accordance with one embodiment of the invention.

FIG. 5 is a side view of the wearable sanitizing dispenser device in operation, in accordance with one embodiment of the invention.

FIG. 6 is a side view of the wearable sanitizing dispenser device, in accordance with another embodiment of the invention.

### DETAILED DESCRIPTION OF THE INVENTION

While the specification concludes with claims defining the features of the invention that are regarded as novel, it is believed that the invention will be better understood from a consideration of the description in conjunction with the drawings. As required, detailed embodiments of the present invention are disclosed herein; however, it is to be understood that the disclosed embodiments are merely exemplary of the invention which can be embodied in various forms. Therefore, specific structural and functional details disclosed herein are not to be interpreted as limiting, but merely as a basis for the claims and as a representative basis for teaching one skilled in the art to variously employ the inventive arrangements in virtually any appropriately detailed structure. Further, the terms and phrases used herein are not intended to be limiting but rather to provide an understandable description of the invention.

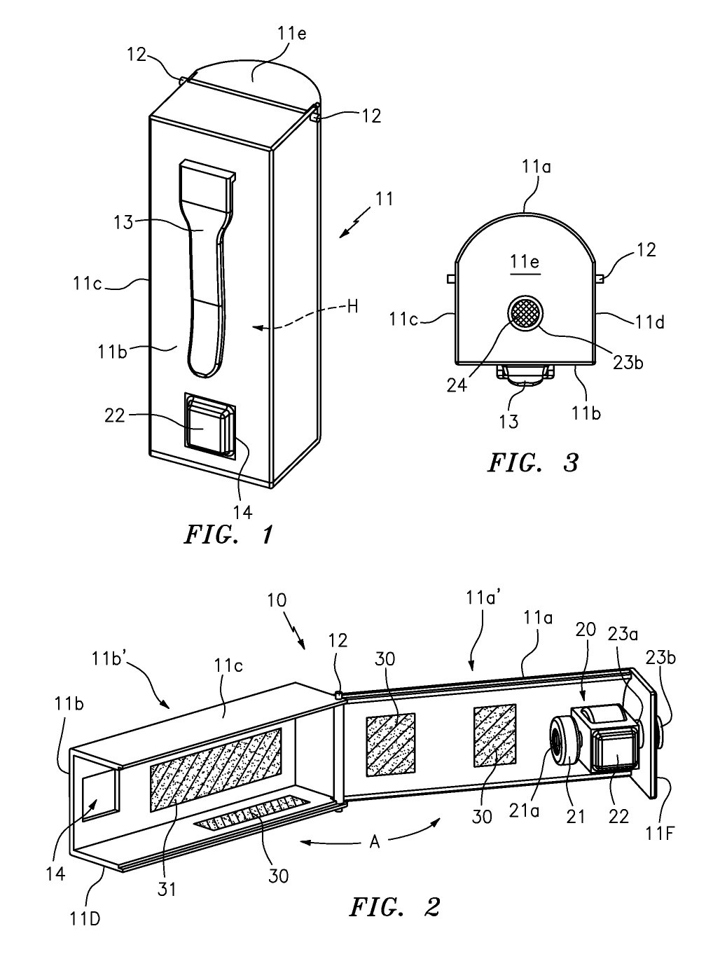

Identical reference numerals are used for like elements of the invention or elements of like function. For the sake of clarity, only those reference numerals are shown in the individual figures which are necessary for the description of the respective figure. For purposes of this description, the terms “upper,” “bottom,” “right,” “left,” “front,” “vertical,” “horizontal,” and derivatives thereof shall relate to the invention as oriented in FIG. 1.

Owing to the rigors of work performed by first responders such as police, firemen and EMT's, for example, conventional pressurized hand sanitizers can not be utilized in a portable (i.e., away from a vehicle) manner, due to the risks of the contents exploding when exposed to extreme heat (firemen) and extreme impacts (police engaged in a physical confrontation). As such, FIGS. 1-3 illustrate one embodiment of a wearable sanitizing agent dispenser 10 that is useful for understanding the inventive concepts disclosed herein. As shown, the device can include a durable wearable holster 11 configured to store a canister 40 capable of dispensing a sanitizing agent 5 to a user on the go.

The holster 11, according to one embodiment can include a front wall 11a, a back wall 11b, a pair of opposing side walls 11c and 11d, a top wall 11e and a bottom wall 11f, each defining a generally hollow interior space H. A belt clip 13 can be disposed along the upper portion of the back wall 11b, and an opening 14 for receiving the dispenser button 22 can be located near the bottom of the back wall 11b.

In one preferred embodiment, the holster 11 can be divided into a pair of complementary halves configured to rotate about a central hinge 12 from a closed position (FIG. 1) to an open position (FIG. 2), see arrow A. To this end, the front wall 11a, the top wall 11e, and the bottom wall 11f can include an integral construction referred to herein as the front half 11a′ of the holster, and the back wall 11b, and opposing side walls 11c-11d can also include an integral construction referred to herein as the back half 11b′ of the holster.

A dispenser 20 can be secured onto the inside facing surface of the bottom wall 11f, within the interior space H. In one embodiment, the dispenser 20 can include a dispensing button 22 that is interposed between a canister connector 21 and an outlet chamber 23.

As described herein, the connector 21 can act to securely engage a canister 40, as described below in order to receive a sanitizing agent 5 that is stored therein. To this end, the connector 21 can include a generally tubular member having a plurality of integrated threads 21a for receiving the canister 40 via a twisting motion. Of course, any number of other devices capable of allowing the connector end 21 to receive a canister in a removable manner can also be utilized in addition to, or instead of the plurality of threads.

The outlet 23 can include a hollow tubular member having a first end 23a connected to the valve, and a second end 23b that traverses/passes through the bottom wall 11f so as to discharge the received agent from the bottom of the device. End 23b can be flush with the bottom wall 11f, or may extend beyond the wall.

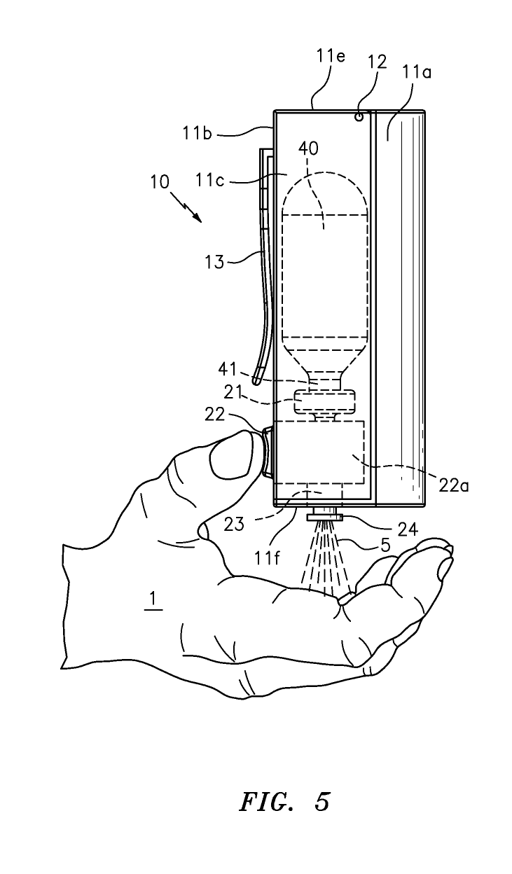

A dispensing valve 22a (See FIG. 5) can be interposed between the canister connector 21 and the outlet chamber 23, so as to control the flow of the sanitizing agent from the canister 40 through the exit of the device. The valve 22a can be controlled by a dispensing button 22 in a conventional manner to pump the sanitizing agent from the canister 40 through the chamber 23. Dispensing valves of this type are well known in the art, and may include a spring (not illustrated) to assist with the pumping action.

Although described above as including separate elements, those of skill in the art will recognize that each of the connector 21, valve 22 and outlet 23 can be constructed as a single unit in accordance with known construction methodologies. Additionally, the dispenser 20 can be positioned virtually anywhere within the interior space H and the outlet chamber can pass through any wall of the holster. Moreover, although described above as a button 22, those of skill in the art will recognize that any device capable of controlling the dispensing valve 22a can be utilized. Several examples include, twist knobs, levers and the like.

In another embodiment, a screen 24 can be positioned within the outlet chamber 23, in order to transition a received sanitizing agent 5 from a gel state into a foam state. In one preferred embodiment, the screen 24 can include a series of narrow cross mounted and/or overlapping vanes disposed within the tubular body of the outlet chamber 23.

The holster 11 can act as a vessel for securely positioning a canister 40 containing a sanitizing agent, and can be constructed from rugged and durable material such as plastic, PVC or other light-weight, impact resistant polymers. Of course other materials ranging from composites to hardened metal are also contemplated. As the device is intended for use by first responders, it is preferred that the components are as durable as possible, so as to withstand any number of direct physical impacts and high temperature variations without breaking or inadvertently dispensing the canister contents. Such instances are desirable for police who may wish to clip the dispenser onto their utility belt, for example. Although described above as including a specific shape, those of skill in the art will recognize that the holster can comprise any number of different shapes and configurations suitable for performing the inventive concepts disclosed herein, without undue experimentation.

In one alternate embodiment, the holster can further include one or more strips of pliable, impact absorbing liners 30, such as rubber, for example, to provide additional protection to the canister and sanitizing agent against damage caused from physical impacts. Additionally, one or more strips of insulating material 31 such as neoprene and/or polyester, for example, can be included to protect the canister and sanitizing agent against damage caused from extreme temperature variations. Each of the liners 30 and insulators 31 can be positioned throughout part of or the entirety of the interior sections of the holster walls in order to provide protection, as described above.

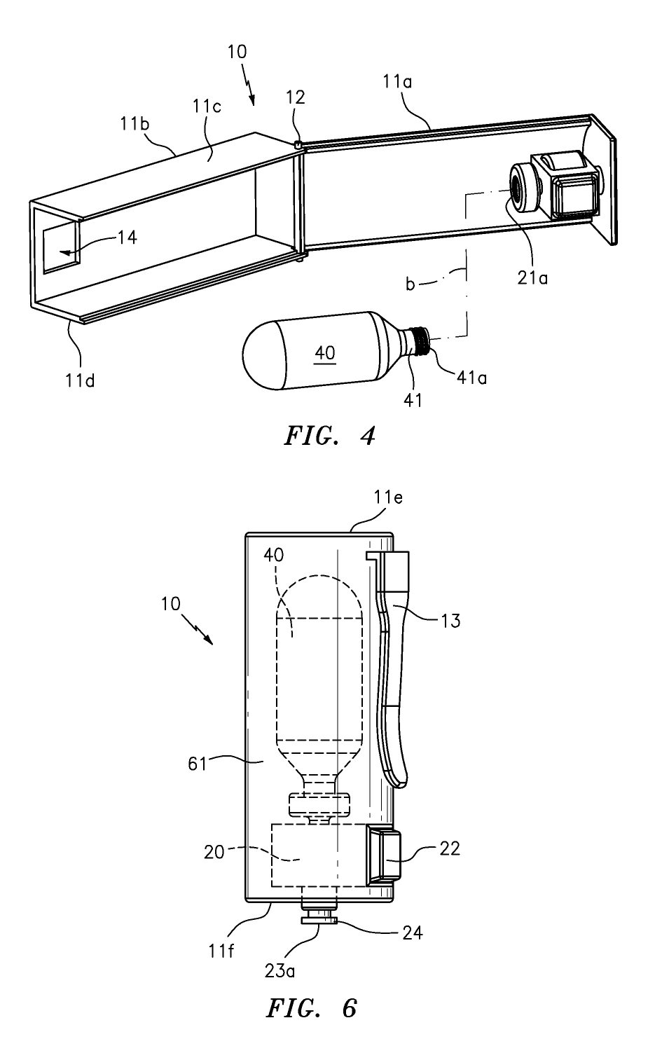

FIG. 4 illustrates one embodiment of a canister 40 which can be removably secured to the dispenser 20 and stored within the holster 11. As shown, the canister can include an open top end 41 having a plurality of threads 41a which are complementary to the threads 21a of the canister connector, so as to allow the canister to be secured to, and removed from the dispenser, see arrow b, via a twisting motion.

In one preferred embodiment, the canister will be constructed from injection molded plastic and include a shape and dimension suitable for being housed within the hollow interior space of the holster in a closed position. Of course, those of skill in the art will recognize that the canister can be of any shape, size or material capable of being housed in the interior space of a holster. It is preferred that the canister be non-pressurized so as to ensure that the canister can not rupture and explode if/when exposed to any extreme impacts which the device may receive during the daily operations of a first responder. However, it is contemplated that some embodiments may utilize canisters having CO₂ and other gas pressurized type devices capable of discharging aerosol, liquid and/or gel solutions via the dispenser. Each of these devices is well and truly known in the art, therefore no further description will be provided.

The canister 40 will act as a vessel for providing a sanitizing agent 5 to the dispenser. Sanitizing agents are well known in the art and typically include one or more active ingredients such as isopropanol, ethanol, n-propanol, or povidone-iodine, as well as inactive ingredients such as polyacrylic acid, humectants, propylene glycol, for example, that are designed to combat the spread of germs and diseases. These agents can take the form of a liquid, gel, foam (when contained in a pressurized canister). Moreover, as described above, by including the screen 24, the dispenser can churn a gel sanitizer into foam for use by the first responder. The specifics of how and why sanitizing agents can transition from a gel state to a foam state when pushed through a screen are outside the scope of this invention, however it should be sufficient to note that the screen allows aeration of the gel which reduces the weight and density of the agent, allowing it to form a more foam-like consistency.

FIG. 5 illustrates one embodiment of the wearable sanitizing agent dispenser 10 in operation. As shown, once the canister 40 is secured within the device, the sanitizing agent 5 can be dispensed through the bottom wall 11f of the device when activated by the button 22. Additionally, by placing the dispenser button 22 on the back wall 11b, the shape of the device itself can act to prevent accidental discharge of the solution 5 resulting from inadvertent contact with foreign objects. To this end, the button 22 will be protected by the body of the wearer, when the device is secured to a belt via the belt clip 13. Moreover, by including the button along the bottom end of the back wall, the users hand 1 can make a natural cupping motion to receive the solution 5 when pressing the button 22.

FIG. 6 illustrates an alternate embodiment of the wearable sanitizing agent dispenser 10 having a non-removable cartridge 40 wherein the entire device is configured to be discarded when the contents of the canister have been exhausted. In this embodiment, the holster 11 can include a generally tubular shape having a side wall 61 that is permanently secured to each of the top wall 11e, and the bottom wall 11f in accordance with known construction methodologies. The dispenser 20 can be secured to the bottom wall 11f, as described above, and the cartridge 40 can be permanently secured to, or incorporated into the construction of the dispenser 20 at a time of manufacture. Such a feature can provide options for achieving the inventive concepts disclosed herein at a lower manufacturing cost.

As described herein, one or more elements of the sanitizer agent dispenser 10 can be secured together utilizing any number of known attachment means such as, for example, screws, glue, compression fittings and welds, among others, and in accordance with known construction methodologies.

As to a further description of the manner and use of the present invention, the same should be apparent from the above description. Accordingly, no further discussion relating to the manner of usage and operation will be provided.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

What is claimed is:

1. A wearable sanitizing dispenser device, comprising:
a holster having a plurality of walls forming a front half and a rear half, and defining a generally hollow interior space, said front and rear halves being hingedly connected together to provide access to the interior space; and
a dispensing unit located within the interior space, said dispensing unit including
a canister connector configured to engage a removable canister stored within the hollow interior space,
an outlet chamber having a portion that traverses a wall of the holster,
a dispensing valve that is in communication with the connector and the outlet chamber, and
a button configured to control the dispensing valve,
wherein the dispensing unit is configured to receive a sanitizing agent contained within the canister, and discharge the same to a user via the outlet chamber.

2. The device of claim 1, wherein the canister connector includes a plurality of threaded elements configured to engage complementary threaded elements located on the removable canister.

3. The device of claim 1, further comprising:
a screen disposed within the outlet chamber, said screen being configured to transition a received sanitizing agent from a gel state to a foam state.

4. The device of claim 1, wherein the holster is constructed from a rugged lightweight material configured to protect a removable cartridge located within the interior space, and connected to the connector from physical damage.

5. The device of claim 1, further comprising:
padding that is positioned within the interior space of the holster, said padding being configured to protect the canister against damage caused from physical impacts.

6. The device of claim 1, further comprising:
insulation that is positioned within the interior space of the holster, said insulation being configured to protect the canister against damage caused from extreme temperature variations.

7. The device of claim 1, wherein the outlet chamber traverses a bottom wall of the holster, and the button is positioned along a back wall of the holster, said button and outlet chamber being configured to accommodate a natural cupping motion of a users hand to operate the device.

8. A wearable sanitizing dispenser device, comprising:
a holster having a plurality of walls forming a front half and a rear half, and defining a generally hollow interior space, said front and rear halves being hingedly connected together to provide access to the interior space;
a removable canister disposed within the interior space;
a sanitizing agent disposed within the removable canister; and
a dispensing unit located within the interior space, said dispensing unit including
a canister connector that is in communication with the removable canister stored within the hollow interior space,
an outlet chamber having a portion that traverses a wall of the holster,
a dispensing valve that is in communication with the connector and the outlet chamber, and
a button configured to control the dispensing valve,
wherein the dispensing unit is configured to receive the sanitizing agent contained within the canister, and dispense the received agent to a user via the outlet chamber.

9. The device of claim 8, further comprising:
a screen disposed within the outlet chamber, said screen being configured to transition the received sanitizing agent from a gel state to a foam state.

10. The device of claim 8, further comprising:
padding that is positioned within the interior space of the holster, said padding being configured to protect the canister against damage caused from physical impacts.

11. The device of claim 8, further comprising:
insulation that is positioned within the interior space of the holster, said insulation being configured to protect the canister and the sanitizing agent against damage caused from extreme temperature variations.

12. The device of claim 8, wherein the outlet chamber traverses a bottom wall of the holster, and the button is positioned along a back wall of the holster, said button and outlet chamber being configured to accommodate a natural cupping motion of a users hand to operate the device.

13. A wearable sanitizing dispenser device, comprising:
a holster having a plurality of walls defining a generally hollow interior space;
a non-pressurized canister disposed within the interior space;
a sanitizing agent disposed within the canister; and
a dispensing unit located within the interior space, said dispensing unit including
a canister connector that is permanently secured to the canister stored within the hollow interior space,
an outlet chamber having a portion that traverses a wall of the holster,
a dispensing valve that is in communication with the connector and the outlet chamber, and
a button configured to control the dispensing valve,
wherein the dispensing unit is configured to receive the sanitizing agent contained within the canister, and dispense the received agent to a user via the outlet chamber.

14. The device of claim 13, further comprising:
a screen disposed within the outlet chamber, said screen being configured to transition the received sanitizing agent from a gel state to a foam state.

15. The device of claim 13, further comprising:
padding that is positioned within the interior space of the holster, said padding being configured to protect the canister against damage caused from physical impacts.

16. The device of claim 13, further comprising:
insulation that is positioned within the interior space of the holster, said insulation being configured to protect the canister and the sanitizing agent against damage caused from extreme temperature variations.

17. The device of claim 8, wherein the outlet chamber traverses a bottom wall of the holster, and the button is positioned along a back wall of the holster, said button and outlet chamber being configured to accommodate a natural cupping motion of a users hand to operate the device.
